(19) United States
(12) Patent Application Publication
(10) Pub. No.: US 2002/0198498 A1
(43) Pub. Date: Dec. 26, 2002

(54) Syringe for high-viscosity fluids

(76) Inventors: David Porat, Newton, MA (US); Harold J. Kosasky, Chestnut Hill, MA (US)

Correspondence Address: MORSE, ALTMAN & MARTIN, P.O. BOX 6926, BOSTON, MA, 02102, US

(21) Appl. No.: 10/198,381

(22) Filed: Jul. 18, 2002

(51) Int. Cl. A61M 5/00

(52) U.S. Cl. 604/187

(57) ABSTRACT

A syringe for high-viscosity fluids that includes a tubular barrel with an opening at one end, a tip at the other end, and a wall therebetween, and a reciprocating plunger with head at one end. The barrel wall includes an aperture that permits filling the syringe with a high-viscosity fluid. To facilitate smooth passage of the plunger head past the aperture and to reduce the chance of user injury, the aperture edges and corners are rounded. The tip has an orifice that is shaped to minimize adherence of the fluid with inner and outer parallel surfaces and an outer surface that curves shallowly all around to the edge.

### CROSS-REFERENCES TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISK APPENDIX

Not Applicable

### BACKGROUND OF THE INVENTION

1. Field of the Invention

The present invention relates to medical testing, more particularly, to a syringe for use with high-viscosity fluids.

2. Description of the Related Art

Fluids, particularly body fluids, come in a wide range of viscosities, from very low viscosities, such as water, to very high viscosities, such as meconium, the first bowel movement of a newborn. When testing fluids, it is important that the fluid remain uncontaminated, or the test results will not be valid. Thus, some tests recommend the use of a syringe for taking, transporting, and depositing fluid samples on test instruments.

A syringe of the prior art is filled by pushing the plunger completely into the barrel, inserting the tip into the filling fluid, and pulling the plunger from the barrel. The small vacuum created by pulling the plunger from the barrel causes the fluid to be sucked through the tip into the barrel. With high-viscosity fluids, however, the vacuum created by pulling the plunger is typically not large enough to force the high-viscosity fluid to deform enough to be pulled through the barrel tip. Thus, other means must be found to fill the syringe.

A solution to filling the syringe with a high-viscosity fluid is to remove the plunger completely from the barrel, stuff the fluid into the opening, and then reinsert the plunger. There are at least two problems with this method. First is the mess. The finger grips for operating the syringe would most likely be smeared with the fluid, not only causing a mess, but also possibly making the syringe slippery. Second, contamination of the fluid is a concern. The typical barrel has an annular ring on its inner surface to prevent inadvertent removal of the plunger. The user must use a relatively large force to pull the plunger from the barrel. When the plunger is released, it pops from the barrel in an uncontrolled manner. When reinstalling the plunger, it must be aligned precisely with the barrel opening before being pushed in, all while the fluid is massed around the opening. Either of these operations can lead to contamination of the fluid, either through contamination of the fluid directly, or by contamination of the plunger tip that makes contact with the fluid.

### BRIEF SUMMARY OF THE INVENTION

An object of the present invention is to provide a syringe that can be used to deposit relatively high-viscosity fluids while avoiding contamination.

The present invention is a syringe for high-viscosity fluids that includes a tubular barrel with an opening at one end, a tip at the other end, and a wall therebetween, and a plunger having a shaft with a head at one end and a pad at the other. The plunger reciprocates axially within the barrel by pulling and pushing the pad. The head makes contact with and propels the fluid in the barrel, and forms a seal with the inner surface of the wall so that the fluid does not leak past the head. The plunger has three positions, (1) the fill position, where the plunger is pulled its maximum extension from the barrel, (2) the prepared position, where the plunger is pushed into the barrel so that the fluid is ready to exit the orifice, and (3) the voided position, where the plunger is pushed as far as it can go into the barrel, ejecting the fluid.

The barrel wall includes an aperture that allows the user to fill the syringe with high-viscosity fluids. The arc of the aperture around the circumference of the barrel cannot be so great that the barrel integrity is compromised. The aperture cannot be so large that the barrel between the aperture and tip cannot hold enough fluid adequate for whatever test will be performed on the fluid after being deposited by the syringe. To facilitate smooth passage of the plunger head past the aperture while reciprocating in the barrel and to reduce the chance that a user will be cut on an edge, the aperture edges are rounded or otherwise blunted. Also, the corners where the edges meet are rounded.

The orifice in the barrel tip is designed to minimize adherence of the fluid to the tip so that the fluid flows smoothly from the tip. The inner surface and outer surfaces of the tip are substantially parallel all around, and the outer surface curves shallowly all around to the edge. The tip edge is dull enough that it will not cause injury to the user. Optionally, the tip is coated with or composed of a non-stick material

Other objects of the present invention will become apparent in light of the following drawings and detailed description of the invention.

### BRIEF DESCRIPTION OF THE DRAWINGS

For a fuller understanding of the nature and object of the present invention, reference is made to the accompanying drawings, wherein:

FIG. 1 is a perspective view of a syringe of the present invention;

FIG. 2 is a cross-section of the syringe of FIG. 1 in the fill position;

FIG. 3 is a cross-section of the syringe of FIG. 1 in the prepared position;

FIG. 4 is a cross-section of the syringe of FIG. 1 in the voided position;

FIG. 5 is an enlarged view of the aperture of the syringe of FIG. 1;

FIG. 6 is an enlarged cross-section of the distal aperture edge of FIG. 2 showing one edge shape;

FIG. 7 is an enlarged cross-section of the proximal aperture edge of FIG. 2 showing another edge shape; and

FIG. 8 is an enlarged cross-section of the tip of the syringe of FIG. 2.

### DETAILED DESCRIPTION OF THE INVENTION

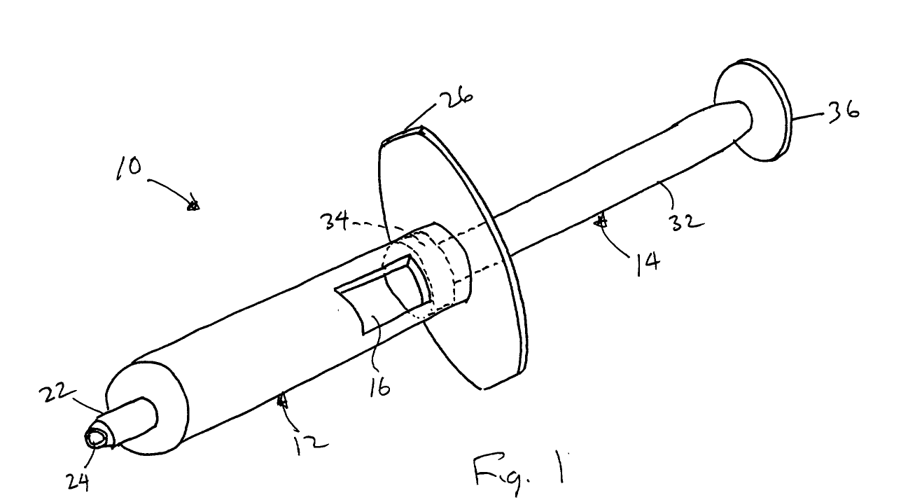

The syringe 10 of the present invention is shown in FIG. 1. It is based on syringes of the prior art and includes a barrel 12 and a plunger 14. The barrel 12 is tubular with an opening 20 at one end and a tip 22 with an orifice 24 at the other end. The barrel 12 has a finger grip that is typically a pair of opposed tabs 26 extending perpendicularly from the rim of the barrel opening 20. Optionally, the barrel 12 includes graduations 28 to indicate the quantity of fluid in the syringe 10 that are typically marked in milliliters (ml).

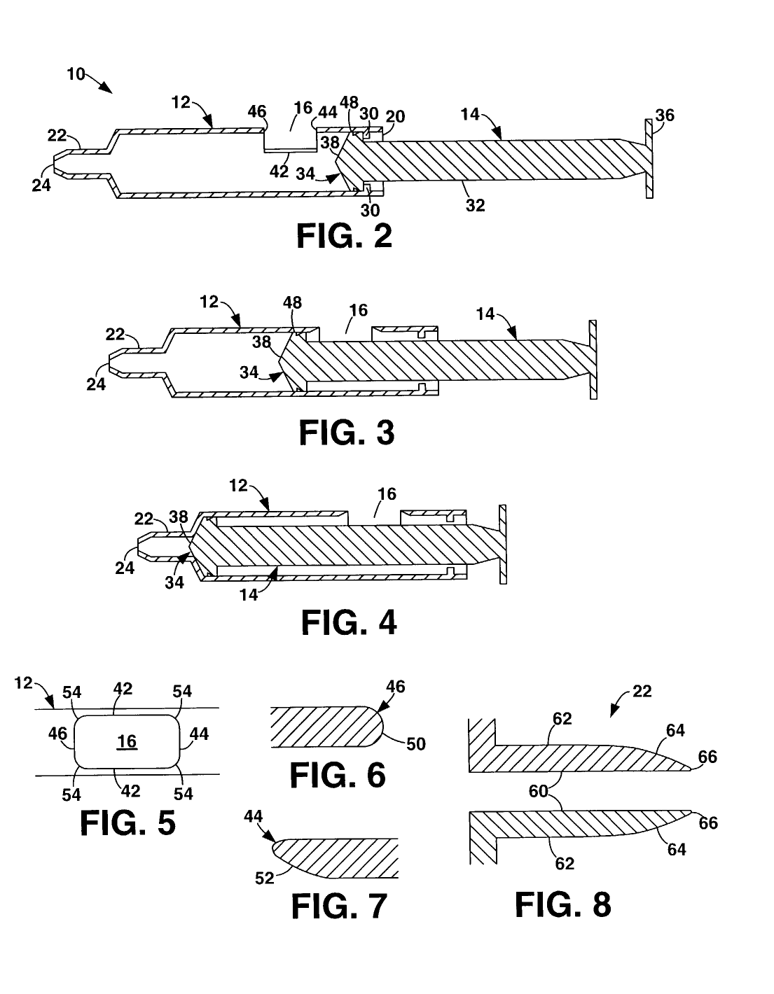

The plunger 14 reciprocates axially within the barrel 12. The plunger 14 has a shaft 32 with a head 34 at one end and a pad 36 at the other. The face 38 of the head 34 makes contact with and propels the fluid in the barrel 12. In some configurations, the head 34 is composed of a resilient material, such as a rubber, that forms against the inner surface of the barrel 12 to provide a seal 48. In other configurations, the head 34 is formed with tolerances tight enough to provide a seal without resiliency. The seal (1) substantially prevents air from leaking past the head 34 into the barrel 12 during suction and (2) substantially prevents the fluid from leaking past the head 34 out of the barrel 12 during injection. The pad 36 is pulled and pushed in order to cause the plunger 14 to reciprocate within the barrel 12 between three basic positions: (1) the fill position, (2) the prepared position, and (3) the voided position. In the fill position, shown in FIG. 2, the plunger 14 is pulled from the barrel 12 to the point of being stopped by an annular ring 30 within the barrel 12. In other words, the plunger 14 is at its maximum normal extension from the barrel 12. In the prepared position, shown in FIG. 3, the plunger 14 is pushed into the barrel 12 to the point where the volume of space in the barrel 12 between the tip 22 and the plunger head 34 is at least large enough to hold enough fluid for the application. At this point, the plunger head 34 has pushed the fluid to fill the barrel 12 adjacent to the tip 22. In the voided position, shown in FIG. 4, the plunger 14 is pushed as far as it can go into the barrel 12 and the majority of the fluid has been ejected from the syringe 10 through the orifice 24.

The barrel 12 and plunger 14 are composed of materials well-known in the art, for example, polyethylene. Optionally, the barrel is composed of or coated with a non-stick material so that the high-viscosity fluid is ejected more easily. Such materials are well-known in the art, and include polystyrene, silicone, and polytetrafluoroethylene.

One aspect of the present invention is the inclusion of an aperture 16 in the wall of the barrel 12 to allow a user to fill the syringe 10 with high-viscosity fluids. The surface area of the aperture 16 is large enough to allow the user to easily insert a quantity of fluid into the barrel 12 sufficient for the task. There are two restrictions that effect the maximum surface area of the aperture 16. First, the arc between the two side edges 42 of the aperture 16 through which the aperture 16 extends around the circumference of the barrel 12 must be small enough so that the barrel 12 does not loose integrity, bend at the aperture 16 under a slight amount of side pressure, or prevent the plunger 14 from being pushed out of the barrel 12 through the aperture 16. It is preferred that the arc be less than 180°. However, if the barrel 12 wall is thick enough to prevent integrity issues, the arc may be greater than 180°.

The second restriction affecting the surface area of the aperture 16 is its location. During use, the syringe 10 will be filled when the plunger 14 is in the fill position of FIG. 2. Therefore, the proximal edge 44 of the aperture 16 cannot be closer to the barrel opening 20 than the plunger face 38 when the plunger 14 is in the fill position.

The distal edge 46 of the aperture 16 is restricted in how close it can be to the tip 22 by the amount of fluid that the syringe 10 needs to hold. The maximum amount of fluid that can be held by the syringe 10 is dictated by the inside volume of the barrel 12 between the tip 22 and the face 38 when the plunger head 34 is closer to the tip 22 than the aperture 16 and forming a seal with the barrel 12, as in FIG. 3. This is also the prepared position when the syringe 10 is filled to capacity. Thus, the distal edge 46 of the aperture 16 cannot be closer to the tip 22 than the location of the seal 48 when the plunger 14 is positioned to enclose the maximum amount of fluid in the barrel 12.

When using a plunger head 34 of a resilient material, the seal 48 between the head 34 and barrel 12 is formed by making the outside diameter of the resilient material slightly larger than the inside diameter of the barrel 12 so that the head 34 is compressed within barrel 12. As the head 34 passes the aperture 16, there is no barrel surface to compress against, so the head expands to its uncompressed shape into the aperture 16. Then, when the head 34 reaches the aperture proximal edge 44 or distal edge 46, depending upon direction of motion, it may be caught, possibly causing the head 34 to be torn or otherwise damaged.

To facilitate smooth passage of the plunger head 34 past the aperture 16 in such cases, the edges 42, 44, 46 of the aperture 16 are shaped to remove any sharp corners and edges in order to minimize the chance that the resilient plunger head 34 will be caught as it moves from the fill position to the prepared position or vice versa. Two such shapes are shown in FIGS. 6 and 7, where the edge 46 is rounded, as at 50, or the edge 44 is rounded and the inner portion is beveled, as at 52. In addition, the corners 54 where the edges meet are rounded. Rounded edges 42, 44, 46 have the added benefit in that the chance that a user will be cut on an edge is greatly reduced.

The size of the orifice 24 depends upon the viscosity of the fluid with which the syringe 10 is expected to be used. In general, the greater the viscosity of the fluid, the larger the orifice 24 needs to be.

Another aspect of the present invention is the shape of the tip 22 at the orifice 24. When a high-viscosity fluid is ejected through a small hole, it has a tendency to curl, sometimes completely back onto the syringe, because of the tendency of the fluids to adhere to solid surfaces and imperfections in the hole itself.

The tip 22 of the syringe of the present invention is shown in detail in FIG. 8. The tip 22 has an orifice 24 that is designed to minimize adherence of the fluid to the tip 22 so that the fluid flows smoothly from the tip 22 to the surface on which it is being deposited. The inner surface 60 of the tip 22 is substantially parallel all around out to a relatively sharp edge 66. The outer surface 62 of the tip 22 is substantially parallel all around and then curves shallowly all around to the edge 66, as at 64. For reasons of safety, the tip edge 66 is dull enough that it will not cause harm under normal use. Optionally, the tip is composed of or coated with a material to which the fluid will not adhere or stick to under normal use.

Generally, the syringe 10 will be used for depositing high-viscosity fluids for testing purposes. Consequently, the syringe 10 should be transported and stored in a manner that will minimize pyrogens inside the syringe 10. To maintain this condition, the syringe 10 will be packaged. Forms of packaging for such purposes are well-known in the art. Example packaging materials includes paper, cellophane, and foil.

### OPERATION

The syringe 10 is first removed from its package and the plunger 14 is pulled out to the fill position. Next, the fluid is procured with a spatula or other small, flat utensil. The utensil is preferably composed of a pyrogen-free material, that is, a material that does not release its own molecules to contaminate the fluid. An adequate amount of the fluid is pushed into the aperture 16 with the utensil making sure that there is an adequate amount of the material in the barrel 12 between the tip 22 and the aperture 38 for the test. The plunger 14 is pushed into the barrel 12 until an adequate amount of fluid for the test has been deposited.

Thus it has been shown and described a syringe which satisfies the objects set forth above.

Since certain changes may be made in the present disclosure without departing from the scope of the present invention, it is intended that all matter described in the foregoing specification and shown in the accompanying drawings be interpreted as illustrative and not in a limiting sense.

We claim:

1. A syringe for a high-viscosity fluid comprising:
(a) a tubular barrel including an opening at one end, a tip with an orifice at the other end, and a wall therebetween, said wall having an inner surface and an outer surface;
(b) a plunger having a shaft with a head at one end, said plunger reciprocating axially within said barrel, said head forming a seal with said wall inner surface;
(c) an aperture in said wall close to said barrel opening.

2. The syringe of claim 1 wherein said orifice has an edge shaped to permit smooth passage of said high-viscosity fluid from said barrel and tip.

3. The syringe of claim 1 wherein said aperture has a circumferential edge with a shape that facilitates smooth passage of said head past said aperture when said plunger is reciprocated within said barrel.

4. The syringe of claim 3 wherein said aperture edge shape includes a bevel between said inner surface and said outer surface.

5. The syringe of claim 3 wherein said aperture edge shape is rounded.

6. The syringe of claim 1 wherein said plunger reciprocates between (a) a fill position wherein said plunger is at its maximum normal extension from said barrel, (b) a prepared position wherein the volume of said barrel between said tip and said plunger head is at least large enough to hold an adequate amount of said fluid, and (c) a voided position wherein said plunger is pushed as far as it can go into said barrel, said aperture being sized so as to fit between locations of said plunger head in said fill position and said prepared position.

7. The syringe of claim 1 wherein said aperture traverses an arc around said barrel that is less than 180°.

8. The syringe of claim 1 wherein said inner surface is non-stick.

9. A syringe for a high-viscosity fluid comprising:
(a) a tubular barrel including an opening at one end, a tip at the other end, and a wall therebetween, said wall having an inner surface and an outer surface, said inner surface being composed of or coated with a non-stick material;
(b) a plunger having a shaft with a head at one end, said plunger reciprocating axially within said barrel, said head forming a seal with said wall inner surface;
(c) an aperture in said wall close to said barrel opening, said aperture having a circumferential edge with a shape that facilitates smooth passage of said head past said aperture when said plunger is reciprocated within said barrel; and
(d) an orifice in said tip having an edge shaped to permit smooth passage of said high-viscosity fluid from said barrel and tip.

10. The syringe of claim 9 wherein said aperture edge shape includes a bevel between said inner surface and said outer surface.

11. The syringe of claim 9 wherein said aperture edge shape is rounded.

12. The syringe of claim 9 wherein said plunger reciprocates between (a) a fill position wherein said plunger is at its maximum normal extension from said barrel, (b) a prepared position wherein the volume of said barrel between said tip and said plunger head is at least large enough to hold an adequate amount of said fluid, and (c) a voided position wherein said plunger is pushed as far as it can go into said barrel, said aperture being sized so as to fit between locations of said plunger head in said fill position and said prepared position.

13. The syringe of claim 9 wherein said aperture traverses an arc around said barrel that is less than 180°.
